(19) United States
(12) Patent Application Publication
(10) Pub. No.: US 2008/0075658 A1
(43) Pub. Date: Mar. 27, 2008

(54) Method of Radiolabeling Formulations for Gamma Scintigraphy Assessment

(76) Inventors: Matthew D. Burke, Durham, NC (US); Jeffrey Scott Staton, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/779,944

(22) Filed: Jul. 19, 2007

Related U.S. Application Data
(60) Provisional application No. 60/807,752, filed on Jul. 19, 2006.

(51) Int. Cl. A61K 51/00; A61P 43/00; G21H 5/00

(52) U.S. Cl. 424/1.29; 252/625; 424/1.73

(57) ABSTRACT

The present invention is directed to a novel method for producing a radiolabeled product for use in gamma scintigraphy, preferably for use with gastric retentive formulations. One aspect of the invention is the process which comprises adsorbing a suitable radionuclide onto a substrate, such as activated charcoal, and blending this nuclide/substrate product with an insoluble polymer; forming a melt blend of the polymer mix, cooling the melt blend to form a solid, and then breaking the solid into smaller particles. Suitably, the temperature of the melt blend is high enough to melt the polymer but not enough to degrade the polymeric material.

### RELATED APPLICATIONS

This application claims the benefit of priority from provisional application U.S. Ser. No. 60/807,752 filed 19 Jul. 2006.

### FIELD OF THE INVENTION

The present invention relates to a method for radiolabeling a gastric retentive formulation for in-vivo imaging studies. More particularly, the present invention relates to a method for radiolabeling gastric retentive formulations for gamma scintigraphy assessment.

### BACKGROUND OF THE INVENTION

Gastric retentive formulations (GRFs) have been pursued by both academia and industry for an extensive period of time, due to the clear benefits of these formulations for drug substances with narrow windows of absorption, for analysis of localized treatment, or for other challenging pharmacokinetic and pharmacodynamic situations. Gastric retentive strategies can be divided into five basic categories: floating, high density, bioadhesive, large size and gastric motility agents.

A significant number of GRFs fall into the category of gastric retention based on expansion/unraveling to obtain a larger size in the stomach than administered and a size that cannot pass through the pylorus. However, the size that a particular object must be in order to be retained in the stomach is not clearly known. Endoscopic data from ingestion of large foreign objects and gastric bezoars indicate that a large, fairly rigid object must be of a size larger than 5 cm in length by 2 cm in diameter in order to be retained for an extensive period of time in the stomach. Endoscopic data also indicate that if the foreign object does not pose an immediate health risk and it is smaller than this size, surgical intervention is not required and the object should pass out of the stomach. Although this is far from a controlled evaluation of the size and strength required to create a GRF, it does provide guidance on the type of size and volumetric expansion of the GRF that is required for gastric retention. To obtain this size and be able to be dosed in a pharmaceutically acceptable format, for example a tablet or capsule, the amount of volumetric swelling is on the order of about 15 times the original size. This is quite a formidable task, but can be achieved with the proper formulation.

The ultimate success of a GRF is based on the pharmacokinetic parameters and appropriate delivery of the drug substance. To determine if a formulation is truly retained in the stomach, a non-invasive approach that does not alter the physical properties of the GRF is preferred. Magnetic resonance imaging is gaining popularity in this area, though such a procedure can be uncomfortable to the patient. Another option is a swallowable camera in the form of a capsule. Video resolution for the swallowable camera is exceptional; however, battery life is limited and controlling the orientation and gastrointestinal transit of the camera is not possible at this time. Gamma scintigraphy has been used extensively for tracking the location of dosage forms in vivo and is often referred to as the “gold standard” for transit studies.

To perform gamma scintigraphy, a small amount of a radioactive element is incorporated in the dosage form, such as a GRF to emit gamma rays. An external device, such as a gamma camera can then track its location in the body.

In order to use a radionuclide to successfully image a GRF using gamma scintigraphy, the radionuclide needs to be retained by the GRF for an extended period of time. This poses a serious challenge depending on the properties of the radionuclide in the gastric environment and the characteristics of the GRF.

While typical radiolabeling approaches have been used successfully for a variety of oral formulations, GRFs provide additional challenges. By design, the GRFs are retained for an extended period of time in a low, yet fluctuating pH environment with compressive mechanical digestive forces. In addition, if gastric retention based on a large size is pursued and a formulation is required to swell 15 or more times its original size, it will often be quite porous in the swollen state, posing further challenges for retention of the radionuclide. Premature leakage of the radiolabel from the formulation may incorrectly suggest gastric emptying or disintegration of the GRF. Other methods, such as absorption onto activated charcoal and Amberlite™ (Rohm & Haas) resin carriers, aqueous based cast films, and conventional bead coating, have all proven to be ineffective when subjected to the harsh conditions of the stomach.

As the current methods presently in use are believed to be ineffective there is a need for adequate labeling of gastric retentive dosage forms, preferably as a radiolabel, that overcomes the physiological challenges described above.

### SUMMARY OF THE INVENTION

The present invention provides a method for producing a novel radiolabel which is incorporated by normal manufacturing processes into a device or formulation which will utilize the radiolabel for determination of location of the device or formulation in a mammal. Suitably, the location is the gastrointestinal tract of the mammal, preferably a human.

The present invention also provides a method for producing a radiolabel, which when incorporated into a device or formulation which utilizes the radiolabel for determination of location in the gastrointestinal tract of a mammal, does not prematurely release the radionuclide due to the compressive and digestive forces of the stomach environment.

The present invention also provides a method for producing a radiolabel, which when incorporated into a device or formulation for determination of location of the device or formulation in the gastrointestinal tract of a mammal, does not prematurely release the radionuclide due to fluctuating pH levels within the stomach environment.

### BRIEF DESCRIPTION OF THE DRAWINGS

FIG. 1 is a view of a GRF which has been radiolabeled with the present invention 18 hours post dose inside a human stomach.

FIG. 2 demonstrates electrospun fibers with a composition of 5.3% SmOx, 47.35% Polyvinylacetate and 47.35% Cellulose Acetate.

FIG. 3 demonstrates an SEM of electrospun fibers with a composition of 95.2% Polycaprolactone and 4.8% SmOx.

FIG. 4 demonstrates an SEM of large beaded electrospun fibers with a composition of 3.2% SmOx 96.8% Polyethylenevinylacetate.

FIG. 5 demonstrates and SEM of electrosprayed beads with a composition of 6.25% SmOx and 93.75% Polyethylene-vinylacetate.

FIG. 6 is a graphic representation of gastric pH fluctuations in a human, throughout the day.

FIG. 7 demonstrates both theoretical and actual measured activity (uCi) of polyethylenvinylacetate SmOx fibers at pH 1.5, having an effective Half Life of 31 hrs.

FIG. 8 (a) demonstrates dissolution at pH 1.5 of a GRF with Indium Chloride/Activated Charcoal/Cellulose Acetate powder incorporated (both Theoretical and measured shown).

FIG. 8 (b) demonstrates dissolution at pH 4.5 of a GRF with Indium Chloride/Activated Charcoal/Cellulose Acetate powder incorporated (both Theoretical and measured shown).

FIG. 9 demonstrates a radiolabled GRF (of Example 1) in the stomach of a mongrel dog 11 hours post-dose. The stomach outline is based on imaging a co-dosed ⁹⁹Technicium labeled egg.

FIG. 10 (a) demonstrates dissolution at pH 1.5 of a GRF with Samarium Oxide powder incorporated (both theoretical and measured).

FIG. 10 (b) demonstrate dissolution at pH 4.5 of a GRF with Samarium Oxide powder incorporated (both theoretical and measured).

FIG. 11 (a) demonstrates dissolution at pH 1.5 of a GRF with Indium Chloride powder incorporated (both theoretical and measured).

FIG. 11 (b) demonstrates dissolution at pH 4.5 of a GRF with Indium Chloride powder incorporated (both theoretical and measured).

### DETAILED DESCRIPTION OF THE INVENTION

The present invention provides a method for producing a novel radiolabel which can be easily incorporated into normal manufacturing processes of a device or formulation which will utilize the radiolabel for determination of location of the device or formulation in a mammal, preferably a human. Suitably, the device or formulation used with the novel radiolabel is a gastroretentive formulation (GRF).

In a specific case described herein, the model GRF chosen is one that can swell to a size large enough to be gastric retentive, thus representing the most difficult case due to the highly porous nature of this swollen GRF. These GRF formulations are described in detail in US Publication No. 20040219186 A1, Nov. 4, 2004, Ayers et al., whose disclosure is incorporated by reference herein in its entirety.

The present invention also provides a method for producing a radiolabel, which when incorporated into a device or formulation which utilizes the radiolabel for determination of location in the gastrointestinal tract of a mammal, does not prematurely release the radionuclide due to the compressive and digestive forces of the stomach environment. It has been reported by Kamba et.al. (2001) Int. J. Pharm. Vol. 228, 209-217 that the force exerted by the stomach on a typical dosage form is approximately 1.5 N in the fasted state and 1.9 N in the fed state.

The present invention also provides a method for producing a radiolabel, which when incorporated into a device or formulation for determination of location of the device or formulation in the gastrointestinal tract of a mammal, does not prematurely release the radionuclide due to fluctuating pH levels within the stomach environment. The typical pH values for the fasted state are 1.0-1.5 and the typical pH values for the fed state can range from a pH of 2.0-5.0 depending on the type and size of the ingested food. Typically, the fed state pH range is 4.0-5.0. Therefore, as food is ingested throughout the day and digested, the pH in the stomach will fluctuate between 1.0 (fasted) to 5.0 (fed), corresponding to the meal size and frequency.

These and other objects and advantages of the present invention are provided by a radiolabeling method that overcomes the presently known challenges of radiolabeling a device or formulation, and provides the ability to track the dosage form throughout the gastrointestinal tract. In one embodiment the dosage form is a GRF.

The method comprises the steps of taking the radionuclide, which is generally available in liquid form, an achieving a powder form of the radionuclide which will not leach out prematurely into the GI fluids, and stays with the device or formulation through its transit in the GI tract. Suitably radionuclide is adsorbed onto a substrate, such as an ion exchange resin or activated charcoal. While these radiolabeled substrates can be used in this manner, as the Working Examples Section demonstrates, they are not ideal as the radionuclide appears to be leaching out of the device or formulation. The present method first takes the liquid radionuclide and adsorbs the nuclide onto the activated charcoal and then admix this radiolabeled substrate with an insoluble polymer. The mixture is then melted, forming a blend of radionuclide and polymer, the melt blend is cooled, forming a brittle solid, which is then broken into smaller particle sizes.

A number of isotopes and polymers can be used in the method of the present invention, as is discussed below.

As noted above, the present invention is directed to method for providing a novel radiolabel which can be incorporated into a variety of devices suitable for tracking gastric motility and, if of interest, gastrointestinal transit. The novel technique provides for encapsulation of a suitable radionuclide and its use in gastric formulations. For purposes of exemplification of the invention a model device, a large gastric retentive formulation was chosen that can swell to a size big enough to be gastric retentive. Alternative formulations which can use this radiolabel include but are not limited to: conventional tablet and multiparticulate formulations, mini-tablets, pharmaceutical films, pharmaceutical hydrogels and xerogels, as well as other types of gastric retentive dosage forms such as floating or high density tablets, multiparticulates, films, electrospun fibers and/or non-woven mats, foams, gels and beads; bioadhesive tablets, multiparticulates, gels, foams, films, and swelling tablets.

To prepare the radiolabel according to the present invention, the first step is to adsorb a radionuclide onto a suitable substrate, in particular activated charcoal or a pharmaceutically acceptable ion exchange resin, such as Amberjet, Amberlite, Duolite, CM-cellulose or DEAE-cellulose.

A suitable radionuclide or isotope for use herein is a nuclide that has an unstable nucleus and decays at a certain half life emitting gamma rays. The radionuclide includes, but is not limited to, indium chloride (¹¹¹InCl), indium, samarium, samarium oxide, technetium, iodine compounds and their derivatives or chelates (such as technetium tin colloid, Pentetate Indium Disodium, etc.) or in compound form. A preferred radionuclide is indium-111, preferably in the form of indium chloride.

As noted, samarium, indium and technetium all represent ideal radionuclide candidates for gamma scintigraphy evaluations. However, for some formulations which require substantial time (˜24 hrs) for manufacturing certain isotopes may not be appropriate, e.g. technetium which has a short half life as compared to indium and samarium. Samarium, as samarium oxide, provides the ability to manufacture using a non-radioactive form of samarium, since samarium can be neutron irradiated alone, or in the final dosage form.

For purposes herein, a “radiolabel” is a product that has a radioactive substance, or radionuclide incorporated in it. The term “radiolabel” shall refer to the final product which is incorporated into a gastric formulation. The present invention may use alternatively use certain terms interchangeably such as radionuclide, isotope, or radioactive substance.

If the radionuclide is adsorbed onto activated charcoal (Sigma Aldrich), it is preferably in a uniform absorption pattern. To ensure this, a small amount of water or acidified water (approximately 0.5 mL per 100 milligrams of charcoal) can be added to the mixture to dilute the radionuclide concentration and allow adequate and uniform exposure of the radionuclide, such as indium to the charcoal, similar to a wet slurry or suspension. If an acid is utilized, it is one which should reduce the pH below a value of 2. Suitable acids include but are not limited to, hydrochloric acid, phosphoric acid or acetic acid. In one embodiment of the invention, the acid is 0.1 N hydrochloric acid.

The activated charcoal with the radionuclide added is dried (also referred to herein as the radiolabeled substrate or radiolabeled charcoal). The most common method of drying is by heating the slurry in a glass vial with a heat gun directed at the bottom of the flask generating a temperature in the flask high enough to evaporate the liquids, e.g. >100° C. Alternatively, other methods, such as drying in an oven, with a hot plate, etc. could be used.

The effective absorbance/retention of indium chloride onto activated charcoal has been evaluated in the range of 50 uCi to 1.2 mCi per 100 mg of activated charcoal.

Suitably, the dried, radiolabeled charcoal is then dry blended with an insoluble polymer in powder form. The blend of polymer and radiolabeled charcoal is heated to a temperature at which it becomes molten, and cooled to a point at which it has a consistency similar to brittle glass. The temperature selected should high enough to melt the insoluble polymer but not enough to degrade the polymeric material. In general this will be above the glass transition temperature (Tg) of the polymer by at least 10° C. It is recognized that each polymer will have a different Tg temperature. For example, when using cellulose acetate as a polymer, which has a melting temperature of 230-300° C., a temperature in or above this range up to 350° C. is suitable to melt the polymer without degrading, when the polymer is exposed to this temperature for a short period of time (up to approximately 5 minutes). The cooled mixture is then ground up into small particles suitable for incorporation into a GRF. Any suitable method for grinding or milling may be used.

The present invention contemplates the use of insoluble polymers which include but are not limited to, cellulose acetate, polyvinylacetate, polyethylvinylacetate, polyethylene, polypropylene, polycaprolactone, polyactic acid, polyglycolic acid, and poly(lactic-co-glycolic acid) (PLGA). A preferred insoluble polymer is cellulose acetate. While it is recognized that enteric polymers can also be used in methods of the present invention, they are not generally preferred as they are more susceptible to the high pH fluctuations that occur in the stomach. Additionally, enteric polymers will not allow complete gastrointestinal transit to be quantified due to dissolution in the intestine.

Suitable weight ratios of radiolabeled charcoal to polymer which can be utilized in the present invention range from about 1:3 to about 1:100. A preferred weight ratio of radiolabeled charcoal to polymer is about 5 to 30, more preferably about 1 to about 6. An example is 0.3 grams of cellulose acetate (Grade CA-398 Eastman Chemicals) to 0.05 grams of radiolabeled activated charcoal.

The particle size of the radiolabel is preferably between about 5 μm and about 20 μm. The particles can be milled to various particle sizes to optimize retention in the GRF or to minimize the impact of the radiolabel on the physical properties of the GRF, as desired. Alternatively, for non-gastric retention applications, the particle could be milled to various sizes and administered separately to investigate particle size effects on transit in the GI tract. In a further embodiment of the invention, for non-gastric retention applications the particles can be nanomilled in order to determine if there is a size below which particles are absorbed through Peyer's patch in the intestine or internalized by endocytosis.

As an alternative substrate, the radionuclide may be placed onto an ion exchange resin (IER) and handled similarly to that of the activated charcoal, but it is not necessary to combine the ion-exchange resin/radionuclide with an insoluble polymer to form a melt blend. Primarily, it is desired to get the radiolabel into a suitable dried state for further manipulation in the device or formulation of choice. For example, indium chloride is supplied in a water solution which is then absorbed onto the charcoal and dried, or alternatively used on an IER instead of the charcoal.

In another embodiment of the invention, the radiolabled particles can be coated with specific compounds that will target specific regions of the body, such as tumor sites. The particles can also be produced with physical characteristics similar to powders used in inhaled or intra-nasal devices, which will provide for investigation of typical flow patterns and deposition in-vivo. The determination of the disposition of drug substance powder by inhaled devices is critical to ensure that the appropriate drug substance reaches the target area and is not deposited in the throat and passed into the stomach, possibly rendering the medication ineffective. The same is true for intra-nasal devices, some of which may target particular regions of the nasal cavity, for example, to target a specific region which may bypass the blood brain barrier to deliver therapeutic agents directly to the central nervous system. Alternatively, this radiolabel product could be coated with mucoadhesive polymers such as chitosan, carbopol, gantrez, etc. to determine if a coating would increase residence time in the nasal cavity.

The ideal photon energy for a radionuclide used in the present invention is between about 100 keV to about 200 keV. Below this range, resolution decreases due to tissue scatter, and above this range sensitivity decreases. Another important aspect of the radionuclide is its half-life. The half-life of the radionuclide will determine the length of time that one can image a radiolabeled formulation. Thus, the longer the half life of a particular radionuclide, the longer a formulation containing that radionuclide can be imaged in a test subject. For example, the half-life of ¹¹¹In is 2.8 days and the photon energy is 247 keV; the half-life of ¹⁵³Sm is 46.27 hrs and the photon energy is 103 keV; and the half-life of ^{99m}Tc is 6.01 hrs and the photon energy is 140 keV.

Indium is an isotope that has a number of optimal characteristics for use in the methods of the present invention. Gastric formulations incorporating indium chloride have been found to work well with gamma scintigraphy analysis under conditions similar to that of the human body. Specifically, indium chloride used with the model GRF formulation exhibited favorable retention at pH levels of about 1.5 and about 4.5. These pH levels represent those exhibited by the human stomach during fasting and after feeding, respectively. Indium also exhibits favorable half-life characteristics making the scintigraphy analysis more effective.

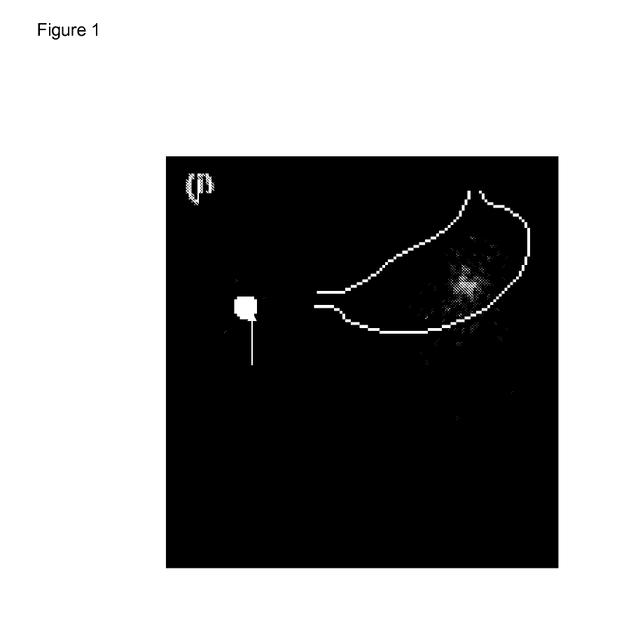

Referring to FIG. 1, a gamma scintigraphy image of a gastric retentive formulation inside a human stomach radiolabeled with indium chloride absorbed onto activated charcoal and enrobed with cellulose acetate is shown. The image in FIG. 1 was made using gamma scintigraphy, taken 18 hours after the radiolabeled GRF was dosed. The fiducial shown in the diagram is an indium-labeled marker used for proper positioning under the scintigraphy camera. In addition, the GRF was co-dosed with a technetium-labeled meal to provide an outline of the stomach. As is shown in the diagram, there is high retention of the radiolabel in the GRF, and virtually no leakage of the radiolabel out of the GRF. This illustrates the advantages of the method of the present invention.

The above mentioned method can also be utilized to enrobe markers for other imaging techniques such as magnetic resonance imaging, or magnetic moment imaging.

Working with radioactivity also limits the type of equipment and scale of equipment normally available for radiolabel manufacture. In addition, manufacture of the radiolabel is often performed at the clinical site the day before dosing. Therefore, such work should only require common equipment available in most labs, while minimizing the time required and minimizing the loss of the activity during manufacture. The present invention only requires a limited amount of lab equipment such as a hot plate and a mortar and pestle.

Another embodiment of the invention is the use of electrospinning to create fibers or beaded fibers which have entrapped nanosized radiolabeled particles, such as SmOx nanoparticles can be used.

Another embodiment of the invention is the use of melt extruded fibers, melt extruded granules, or electrosprayed beads (such as those found in Loscertales et. al. Science, vol. 295, 2002, p. 1695) using other methods well known in the art to create a similar final product with entrapped radionuclides, such as those using a radionuclide which can be irradiated later, e.g., Samarium Oxide. Polymers useable for these methods include those noted above for the herein described polymer melt approach and include but art not limited to: cellulose acetate, polyvinylacetate, polyethylvinylacetate, polyethylene, polypropylene, polycaprolactone, polyactic acid, polyglycolic acid, and poly(lactic-co-glycolic acid) (PLGA). Preferably, the polymers for use in these methods are polyethylene vinyl acetate (PEVAc) and polycaprolactone (PCL).

The electrospinning process may need a suitable solvent, such as an organic solvent. Preferably, the solvent of choice is a GRAS approved organic solvent, or one suitable for obtaining GRAS approval, although the solvent may not necessarily be “pharmaceutically acceptable” one as the resulting amounts may fall below detectable, or set limits for human consumption they may be used. It is suggested that ICH guidelines be used for selection. GRAS in an acronym for “generally recognized as safe”.

Suitable solvents for use herein include, but are not limited to acetic acid, acetone, acetonitrile, methanol, ethanol, propanol, ethyl acetate, propyl acetate, butyl acetate, butanol, N,N dimethyl acetamide, N,N dimethyl formamide, 1-methyl-2-pyrrolidone, dimethyl sulfoxide, diethyl ether, disisopropyl ether, tetrahydrofuran, pentane, hexane, 2-methoxyethanol, formamide, formic acid, hexane, heptane, ethylene glycol, dioxane, 2-ethoxyethanol, trifluoroacetic acid, methyl isopropyl ketone, methyl ethyl ketone, dimethoxy propane, methylene chloride etc., or mixtures thereof. Preferably, the solvent is ethanol, methanol, acetone, ethyl lactate, isopropyl alcohol, dichloromethane, THF and mixtures thereof. The solvent may include aqueous mixtures thereof. A preferred solvent for the polymer PEVAc is THF. A preferred solvent for PCL is 1,1,1,3,3,3-Hexylfluoro-2-propanol in a 60:40 mixture of acetone and ethyl lactate.

The solvent to polymeric composition ratio is suitable determined by the desired viscosity of the resulting formulation. A typical polymer range is 5-10% w/w in the solvent, and the rest of the total volume is organic solvent. For electrospinning of a radiolabeled polymeric composition, key parameters include viscosity, surface tension, and electrical conductivity of the solvent/polymeric composition.

By the term “nanoparticulate” as used herein, is meant, nanoparticule size of the radionuclide within the electrospun fiber, etc.

In another embodiment of the invention the radionuclide can be coated onto a bead, such as a sugar sphere or a microcrystalline cellulose bead, using methods well known in the art to create similar final products with entrapped radionuclides, such as those using a radionuclide which can be irradiated later, such as Samarium Oxide. The beads can be sprayed or produced in a fluidized bed with suitable coating agents premixed with the radionuclide. Suitable coating agents include hydroxypropylmethylcellulose (HPMC) or other suitable cellulosic derivatives. The HPMC is used to adhere the nuclide, e.g. samarium to the sugar sphere as is used in an amount of ˜5% w/w compared to the samarium oxide. The mixture of HPMC and Samarium oxide is applied to the beadlet to achieve a suitable % weight gain in the order of 10-15% w/w. The beadlet is then overcoated with a barrier layer, such as Surelease®, e.g. an ethylcellulose-based coating. The amount of overcoating for the beads is approximately 1.5 times the amount of radionuclide used, for instance samarium oxide on a weight/weight basis.

Methods of Preparation

The invention will now be described by reference to the following examples which are merely illustrative and are not to be construed as a limitation of the scope of the present invention. All temperatures are given in degrees centigrade, all solvents are highest available purity unless otherwise indicated.

### EXAMPLE 1

### Indium Chloride/Activated Charcoal/Cellulose Acetate

Radiolabel Preparation:

50 mg of activated charcoal is weighed into a scintillation vial and to this is added ˜130 uCi of Indium¹¹¹ chloride in solution followed by 1 mL of filtered water to dilute the indium for improved homogeneity of mixture. The mixture is swirled gently, then the water is evaporated using a heat gun until all of the water is removed. The mixture should remain as a powder. The radiolabeled charcoal powder is combined with cellulose acetate (CA-398-10NF) (approximately 300 mg) in a 1:6 ratio and the dry mixture is blended with a spatula to ensure uniformity. The mixture is placed on a hot plate until the mixture melts. The mixture is allowed to cool to a brittle glasslike consistency. The mixture is removed from the container and transferred to a mortar for milling with a pestle until the particle size is approximately 10-20 microns.

Gastric Retentive Dosage Form Preparation:

The GRF is prepared using 1 gram of xanthan gum and 1 gram of locust bean gum dissolved into water using high shear mixing and heat. After dissolution of these polysaccharides, 3 grams of polyethylene glycol 400 is added as appropriate (to ensure flexibility when in the dried state) and the radiolabel preparation (from the preceding example, part 1 above) is added. The aqueous mixture is poured into a mold 1.5 cm×1 cm×7.5 cm and allowed to gel through the formation of physical crosslinks. After gelation, the formulation is placed in an Isotemp vacuum oven Model 282A with ThermoSavant RVT400 refrigerated vapor trap at 50° C. to remove approx 95% of the water. The dried gels are compressed and rolled and placed in a 000 capsule.

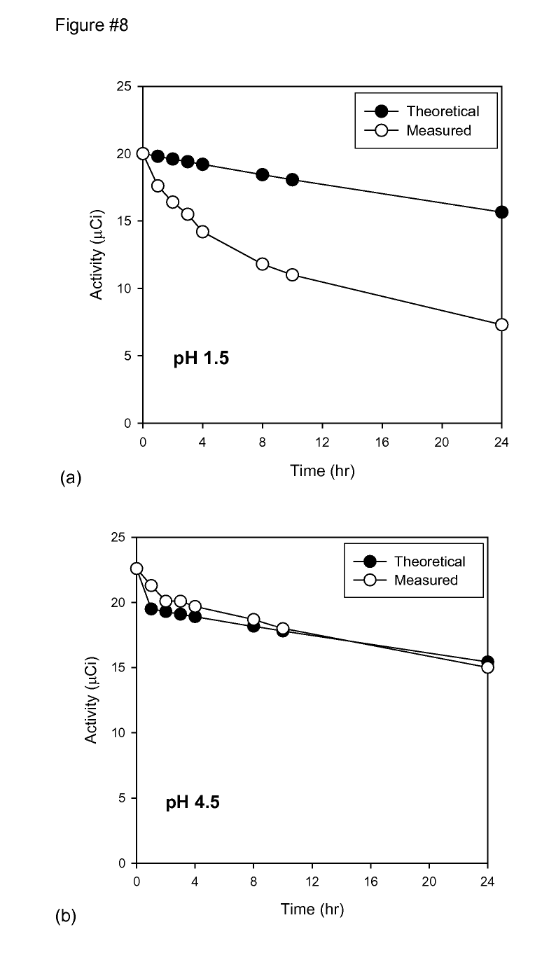

The in-vitro data obtained for this example is shown in FIGS. 8(a) and (b).

FIG. 8 (a) demonstrates dissolution at pH 1.5 of a GRF with Indium Chloride/Activated Charcoal/Cellulose Acetate powder incorporated. The theoretical measurement on this graph is a demonstration of the natural decline in radioactivity of the nuclide over time. Preferably the theoretical measurement and the actual measurement are identical. The difference between the theoretical and the actual measurements in this graph and in FIG. 8 (b) show a loss of radionuclide from the GRF dosage form.

FIG. 8 (b) demonstrates dissolution at pH 4.5 of a GRF with Indium Chloride/Activated Charcoal/Cellulose Acetate powder incorporated.

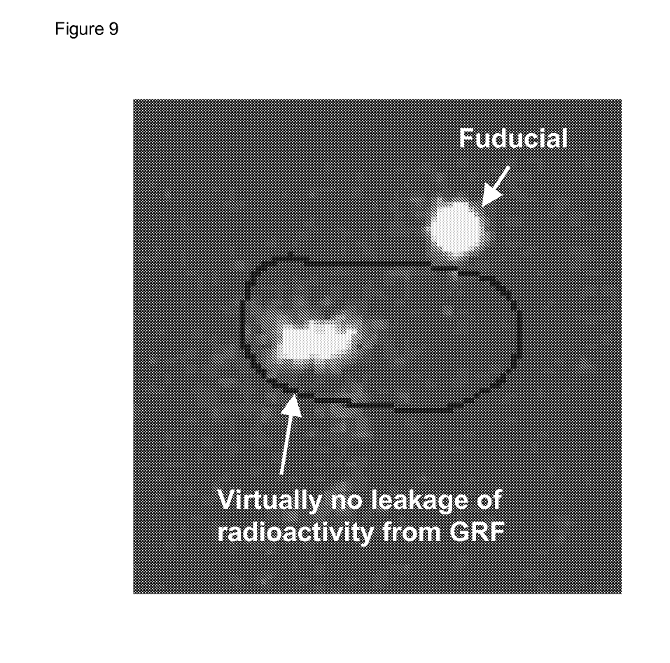

FIG. 9 demonstrates in vivo data of a radiolabled GRF of Example 1, in the stomach of a mongrel dog 11 hours post-dose. The stomach outline is based on imaging a co-dosed ⁹⁹Technicium labeled egg.

Evaluation of the radiolabeled GRF in a large dog model was determined to be potentially a better correlation to man. In previous studies, the beagle dog retained the GRF for a substantial period of time while the performance in man was significantly shorter. This prompted an evaluation in a second large dog model, the foxhound (30-40 kg) versus the beagle (10-15 kg). The foxhound GRF retention continued to be prolonged compared to the performance in man, yet was more predictive than the beagle dog GRF retention. Mongrel dogs (15-20 kg) were used for evaluation of these radiolabeled GRF's, instead of foxhounds. Dosing of the radiolabeled GRF in mongrel dogs revealed virtually no leakage of the radiolabel even after 11 hours post dose as shown in the image of FIG. 9. This result provides confidence that the integrity of the radiolabel is sufficient and reveals that the additional complexity of gastric contractions and digestive actions does not cause premature release of the radiolabel. It has been determined that the pH of the dog's stomach is often elevated in between meals due to a low basal acid secretion rate, and this elevation may provide prolonged retention at elevated pH.

Subsequent evaluation of the GRF in a clinical study (as described herein) revealed that the radiolabel was again highly retained in the GRF. FIG. 1 shows an 18 hour image of a human subject with the GRF still retained in the stomach, and clearly visible with gamma scintigraphy. This figure indicates that virtually no leakage of the radiolabel was observed. The stomach outline is based on imaging a co-dosed ⁹⁹Technicium labeled egg. In fact, based on an initial dosing activity of 0.5 MBq of ¹¹¹In, the location of the GRF could be determined by scintigraphic assessment even beyond the 48 hour timepoint for complete GI transit time estimation.

### EXAMPLE 2

### Samarium Oxide Powder ˜5 um

Radiolabel Preparation:

Samarium oxide powder (˜5 um) was sent to the Missouri University Research Reactor (MURR) nuclear facility and neutron irradiated prior to dosage form preparation. In this example the nuclide was not adsorbed onto charcoal nor an ion-exchange resin.

Gastric Retentive Dosage Form Preparation:

Same as Example 1, except 200 mg of the neutron-activated samarium oxide powder was added to the polyethylene glycol 400 and thoroughly mixed prior to being added to the xanthan gum locust bean gum mixture as the radiolabel. The gels were dried and encapsulated as described in example 1. The encapsulated GRF was then used in in-vitro or in-vivo evaluations.

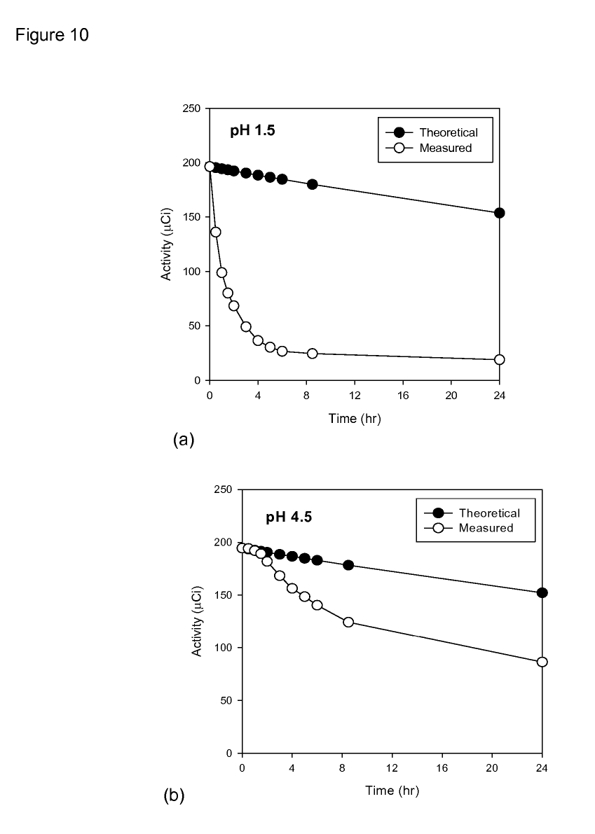

The in-vitro data for this example is shown in FIGS. 10 (a) and (b).

FIG. 10 (a) demonstrates dissolution at pH 1.5 of a GRF with Samarium Oxide powder incorporated.

FIG. 10 (b) demonstrate dissolution at pH 4.5 of a GRF with Samarium Oxide powder incorporated.

Effective Half Life:

| | pH 1.5 | pH 4.5 |
|---|---|---|
| Sm₂O₃ Powder | 0.9 hr | 5.2 hr |

Note:
Sm₂O₃ half life is 46.27 hrs

### EXAMPLE 3

### Samarium Oxide Beads

Radiolabel Preparation:

Sugar spheres (30-35 mesh, JRS Pharma) were coated in a Glatt fluid bed with a samarium oxide/hydroxypropylmethylcellulose mixture to a 13% weight gain, followed by a barrier coat of ethylcellulose (Surelease® E-7-19010, Colorcon) to a 15% weight gain. The samarium oxide beads were neutron irradiated at the Missouri University Research Reactor nuclear reactor facility prior to incorporation into the GRF.

The composition of the SmO beads is listed below. The ratio of HPMC to SmO is 1:19.06.

| Theoretical Bead Content | % wt/wt |
|---|---|
| Sugar starch, NF, Sugar spheres, 30-35 mesh | 76.95 |
| Naturally occurring samarium oxide | 9.53 |
| HPMC E5 | 0.50 |
| Surelease E-7-19010 | 13.04 |
| Total | 100.0 |

Gastric Retentive Dosage Form Preparation:

Using the procedure of example 1 above, a GRF preparation was prepared except using neutron-activated samarium oxide beads (approximately 450 mg) incorporated as the radiolabel.

Using Samarium Oxide Beads the effective half-life, in-vitro was determined to be

| | pH 1.5 | pH 4.5 |
|---|---|---|
| Sm₂O₃ Beads | 3.9 hr | 9.8 hr |

Note:
Sm₂O₃ half life is 46.27 hrs

### EXAMPLE 4

### Electrospinning Cellulose Acetate/Polyvinylacetate/SmOx

Radiolabel Preparation:

This example utilizes another alternative embodiment of electrospinning to create fibers or beaded fibers which have entrapped nanosized SmOx particles. Electrospinning of an active agent, including radiolabels can be found in WO 01/54667 (US2003/0017208) whose disclosure is incorporated herein by reference in its entirety.

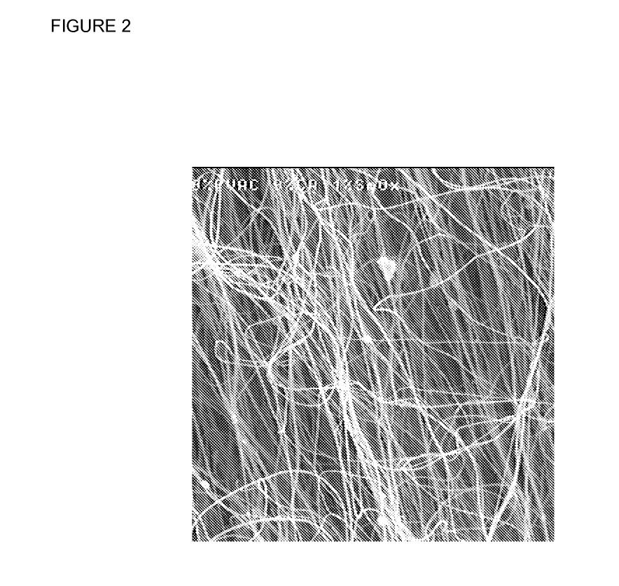

Weigh 2 mL of 60:40 Acetone:Ethyl lactate in a scintillation vial, add 18 mg of cellulose acetate 398-10NF and 18 mg of polyvinylacetate (MW 100,000), stir with a magnetic stir bar until both polymers are dissolved. Add 2 mg of nanomilled Samarium Oxide (Aldrich 637319) and mix until a uniform dispersion is created. Place in a 3 mL syringe equipped with a 20 gauge flat tip needle. Place the syringe in a syringe pump and attach a high voltage cable to the syringe needle. Position a grounded collection plate 24 cm from the end of the syringe needle tip. Begin pumping the solution at a rate of 2.0 mL/hr and turn on the voltage to 17 kV. Electrospun fibers will be created with a final composition of the fibers 5.3% SmOx, 47.35% Polyvinylacetate and 47.35% Cellulose Acetate. Electrospun fibers having this composition are shown in FIG. 2. The in-vitro testing was done solely on the fibers alone and not in a GRF model formulation.

In Vitro Data Demonstrates an Effective Half Life:

| | pH 1.5 |
|---|---|
| Sm₂O₃ CA/PVAc Nanofibers | 0.1 hr |

Note:
Sm₂O₃ half life is 46.27 hrs

### EXAMPLE 5

### Electrospun Fibers Polycaprolactone/SmOx

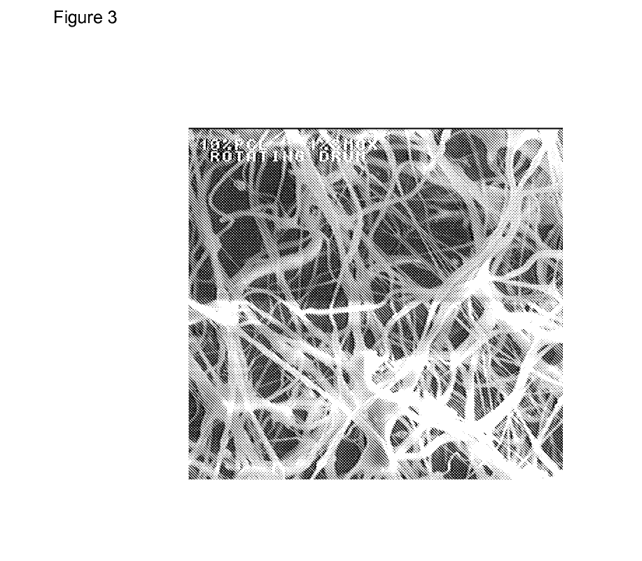

Weigh 2 mL of 60:40 Acetone:Ethyl lactate in a scintillation vial, add 20 mg of polycaprolactone (Sigma), stir with a magnetic stir bar until both polymers are dissolved. Add 2 mg of nanomilled Samarium Oxide (Aldrich 637319) and mix until a uniform dispersion is created. Place in a 3 mL syringe equipped with a 20 gauge flat tip needle. Place the syringe in a syringe pump and attach a high voltage cable to the syringe needle. Position a grounded collection plate 24 cm from the end of the syringe needle tip. Begin pumping the solution at a rate of 1.0 mL/hr and turn on the voltage to 20 kV. Electrospun fibers will be created with a final composition of the fibers 95.2% Polycaprolactone and 4.8% SmOx. An SEM scan of these electrospun fibers is shown in FIG. 3.

The in-vitro data prompted evaluation in the mongrel dog in a similar fashion to example 1. Dosing of a GRF which was radiolabeled with the electrospun fibers of polycaprolactone with SmOx allowed successful evaluation of the location of the GRF in the dog's gastrointestinal tract. The GRF remained in the dog's stomach for approximately 22 hrs in two out of three dogs.

In-Vitro Data Generates an Effective Half Life:

| | pH 1.5 |
|---|---|
| Sm₂O₃ PCL Nanofibers | 8.5 hr |

Note:
Sm₂O₃ half life is 46.27 hrs

### EXAMPLE 6

### Electrospun Fibers Polyethylenevinylacetate/SmOx

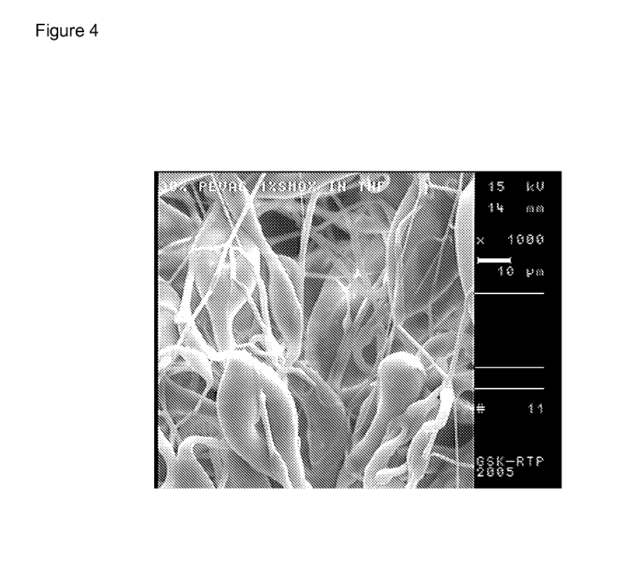

Weigh 2 mL of tetrahydrofuran (THF) in a scintillation vial, add 60 mg of polyethylene vinyl acetate, stir with a magnetic stir bar until both polymers are dissolved. Add 2 mg of nanomilled Samarium Oxide (Aldrich 637319) and mix until a uniform dispersion is created. Place in a 3 mL syringe equipped with a 20 gauge flat tip needle. Place the syringe in a syringe pump and attach a high voltage cable to the syringe needle. Position a grounded collection plate 24 cm from the end of the syringe needle tip. Begin pumping the solution at a rate of 2.0 mL/hr and turn on the voltage to 17 kV. Electrospun fibers will be created with a final composition of the fibers 3.2% SmOx 96.8% Polyethylenevinylacetate. An SEM is shown in FIG. 4 with large beaded fibers.

The in-vitro testing was done solely on the fibers alone and not in a GRF model formulation.

In-vitro data generates an effective Half Life:

| | pH 1.5 |
|---|---|
| Sm₂O₃ PEVAc Nanofibers | 31.0 hr |

Note:
Sm₂O₃ half life is 46.27 hrs

These fibers appear likely to demonstrate successful radiolabel retention with imaging ability at 24 hours plus in an in-vivo model.

### EXAMPLE 7

### Electrosprayed Beads Polyethylenevinylacetate/SmOx

Another alternative process for creating fibers, or beaded fibers, or small beads is by electrospraying. In this Example, ˜15 um beads are manufactured through the process of electrospraying (dry composition: 6.25% SmOx and 93.75% Polyethylenevinylacetate).

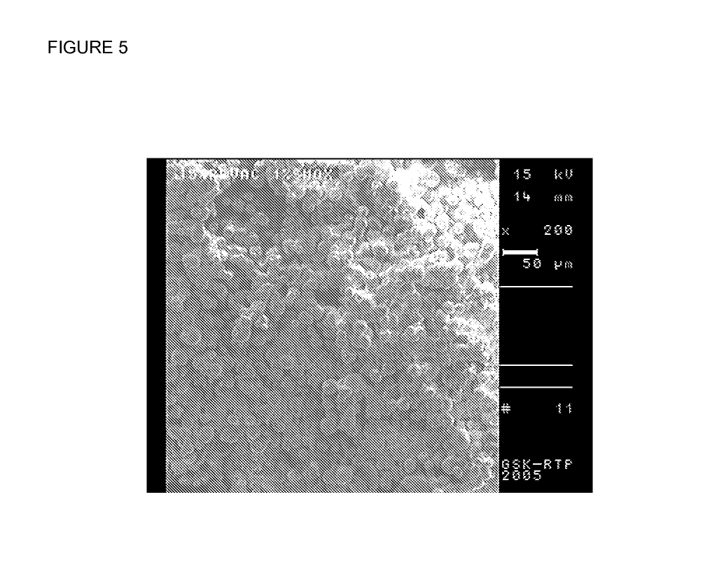

Weigh 2 mL of tetrahydrofuran (THF) in a scintillation vial, add 30 mg of polyethylene vinyl acetate, and stir with a magnetic stir bar until both polymers are dissolved. To this is added 2 mg of nanomilled Samarium Oxide (Aldrich 637319) and mix until a uniform dispersion is created. Place in a 3 mL syringe equipped with a 20 gauge flat tip needle. Place the syringe in a syringe pump and attach a high voltage cable to the syringe needle. Position a grounded collection plate 24 cm from the end of the syringe needle tip. Begin pumping the solution at a rate of 1.5 mL/hr and turn on the voltage to 15 kV. Electrosprayed beads had a final composition of 6.25% SmOx and 93.75% Polyethylene-vinylacetate. A representative SEM is shown in FIG. 5 of the beads.

The in-vitro testing was done solely on the beads alone and not in a GRF model formulation.

In-vitro data generates an effective Half Life:

| | pH 1.5 |
|---|---|
| Sm₂O₃ PEVAc Beads | 30.0 hr |

Note:
Sm₂O₃ half life is 46.27 hrs

### EXAMPLE 8

### Indium Chloride/Activated Charcoal

Radiolabel Preparation:

Weigh 50 mg of activated charcoal into a scintillation vial and add ˜130 uCi of Indium¹¹¹ chloride in solution followed by 1 mL of filtered water to dilute the indium for improved homogeneity of mixture. Swirl gently, then evaporate the water using a heat gun until all of the water is removed. The mixture should remain as a powder.

Gastric Retentive Dosage Form Preparation:

Same as example 1, except using the above radiolabel (Indium Chloride/Activated Charcoal).

In-Vitro:

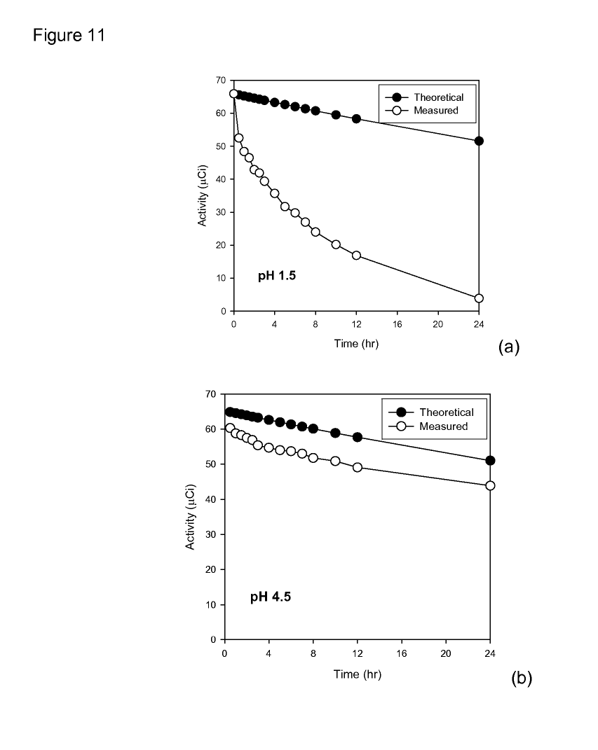

FIG. 11 (a) demonstrates dissolution at pH 1.5 of a GRF with Indium Chloride powder incorporated.

FIG. 11 (b) demonstrates dissolution at pH 4.5 of a GRF with Indium Chloride powder incorporated.

Effective Half Life: Indium Chloride Formulations

| | pH 1.5 | pH 4.5 |
|---|---|---|
| InCl₂ Powder | 5.3 hr | 10.2 hr |

Note:
InCl₂ half life is 67.2 hrs

### EXAMPLE 9

### Indium Chloride/Amberjet 4400

Radiolabel Preparation:

50 mg of Amberjet™ 4400, an ion exchange resin, was weighed into a scintillation vial and to this was added ˜130 uCi of Indium¹¹¹ chloride in solution followed by 1 mL of filtered water to dilute the indium for improved homogeneity of mixture. This is swirled gently, then the water is evaporated using a heat gun until all of the water is removed. The mixture should remain as a powder.

Gastric Retentive Dosage Form Preparation:

Same as example 1, except using the above radiolabel (Indium Chloride/Amberjet 4400)

In-Vitro Data Generates an Effective Half Life for Indium Chloride Formulations:

| | pH 1.5 | pH 4.5 |
|---|---|---|
| InCl₂-Amberjet | 4.1 hr | 25.7 hr |

Note:
InCl₂ half life is 67.2 hrs

Evaluations of some of the above noted examples was determined using the following assays and protocols.

Dissolution Screening

Dissolution is a common technique to characterize the release of a drug substance from a pharmaceutical formulation and is also an effective tool to determine if the radionuclide is successfully retained in a formulation for the amount of time required.

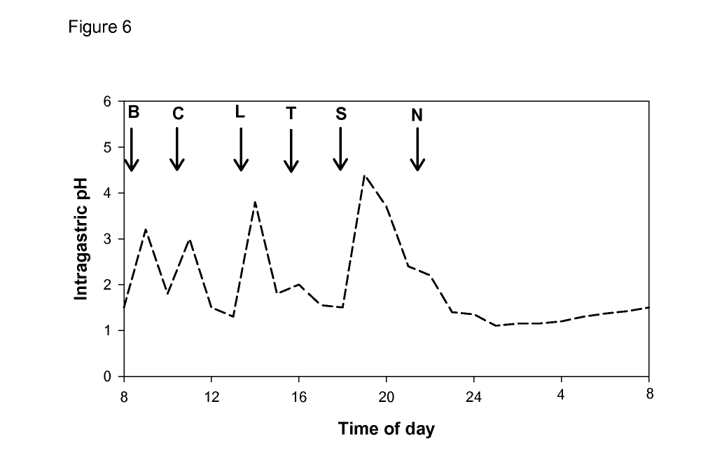

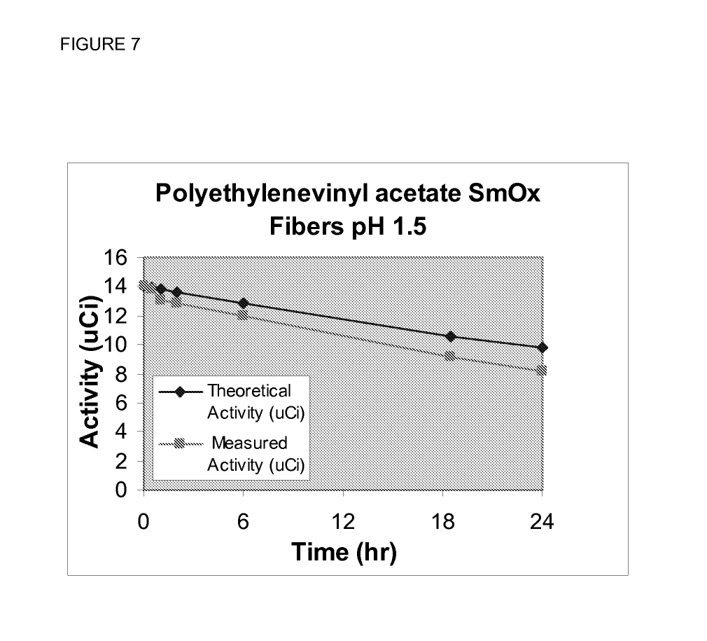

A physiologically relevant pH media should be used when evaluating dissolution. The most common pH to mimic the gastric environment is pH 1.0 or 1.5 to mimic the fasted stomach pH, however, during the fed state the gastric pH can increase to as high as pH 4.5. In addition, the GRF will also be exposed to gastric pH fluctuations throughout the day (See FIG. 6, in Williams et. al. (1998) Aliment. Pharmacol. Ther., Vol. 12, p. 1079-1089). Retention of the radionuclide in the GRF was evaluated at both pH 1.5 and pH 4.5.

The initial radioactivity of the radiolabeled GRF was measured in a Capintec Radioisotope Calibrator® Model CRC-12 and then placed in 500 mL of either pH 1.5 or pH 4.5 media in a Vankel USP II apparatus with a stirring rate of 30 rpm and temperature of 37° C. The GRF was removed at appropriate timepoints and the radioactivity was measured. When the pH was fluctuated, the GRF was physically moved from one dissolution vessel at pH 1.5 to a second vessel at pH 4.5. The higher pH buffer was prepared by adding sodium acetate to a concentration of 25 mM and adjusting the pH to pH 4.5 with 1 M HCL. The low pH buffer was prepared by making a 0.03 N HCL solution with 2% NaCl. No gastric enzymes were used in either dissolution media preparation.

During in-vitro evaluation of the GRF with samarium oxide powder, it was observed that the rate of radiolabel release from the GRF occurred in a similar fashion to an exponential decay process. This is consistent with Fick's second law of diffusion which states that the change in concentration with time in a particular region is proportional to the change in the concentration gradient at that point in the system. This represents a first order process which can be modeled by an exponential function in the ideal case. Therefore, by modeling the radiolabel release from the GRF with an exponential function, an effective half life for the radiolabel in the GRF was obtained. The effective half life is a useful value to access the ability of the radiolabel to be retained in the GRF and to understand how long it is possible to image the GRF in-vivo.

Based on this effective half life it is possible to estimate if there will be enough radiolabel in the GRF at 24 hours for it to be imaged successfully using gamma scintigraphy. Preferably, the minimum requirement for the effective half-life in both pH's should be higher than 10 hours. This provides not only for accurate assessment of GRF performance, but also for safety reasons to ensure the GRF has emptied from the stomach. Preferably, endoscopic procedures will then not need to be performed to investigate the location, and potential removal of the GRF.

Preclinical Assessment of Radiolabel Performance

Male mongrel dogs, similar in weight (17 kg), were housed in individual cages and received a standard diet (Canine food 5006, LabDiet®, IA, U.S.A.) and water ad libitum. The animals were clinically healthy and haematologically and biochemically normal throughout the experimental period. The research adhered to the “Principles of Laboratory Animal Care” (NIH publication #85-23, revised in 1985). Under an approved animal protocol adhering to humane treatment and principles of laboratory animal care, conscious beagles were comfortably seated in a sling, and situated beneath a gamma scintillation camera with the camera head located over the back of the beagle. An e.Cam Fixed 180 dual head SPECT gamma camera (Siemens Medical Solutions, PA, U.S.A.) was equipped with two opposed detectors, each having a 533×387 mm field of view were fitted with low energy parallel hole collimators, and set for dual isotope acquisition.

The ¹⁵³Sm or ¹¹¹In labeled GRF was co-dosed with a ^{99m}Tc labeled liver treat to provide an outline of the stomach. One fiducial (reference marker) was placed on each dog for proper positioning when placing the dog under the camera for image acquisition. Scintigraphic images of 30 seconds duration were simultaneously acquired from both anterior and posterior detectors at 1 hr intervals up to 12 hours and a final image at 24 hrs. Between image acquisitions, the dogs were allowed to move freely in the room or were brought back to their cages. An on-line computer was connected to the camera and digital image recording was performed using an e.Soft programme (Siemens Medical Solutions).

Clinical Assessment of Radiolabel Performance

A single-center, randomized, four-way, within-subject crossover study was performed. The study followed the tenets of the Declaration of Helsinki in 1964 and its subsequent revisions, was approved by the North Glasgow Hospitals University Trust Ethics Committee and the Administration of Radioactive Substances Advisory Committee and was conduced to Good Clinical Practice.

Six healthy male volunteers (age range 35-60 years, inclusive) with a body weight greater than 50 kg and a body mass index (BMI) within the range of 19-29.9 kg/m³ inclusive participated in the study after providing written informed consent. All volunteers were non-smokers, were not taking any medication, had no abnormality on clinical examination, clinical chemistry or haematology examination, and no history of gastrointestinal disease.

The application of which this description and claims forms part may be used as a basis for priority in respect of any subsequent application. The claims of such subsequent application may be directed to any feature or combination of features described herein. They may take the form of product, composition, process or use claims and may include, by way of example and without limitation, one or more of the following claims:

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A process for producing a radiolabeled product, which comprises
a) adsorbing a radionuclide onto activated charcoal;
b) blending the product of step a) with an insoluble polymer in powder form to form a mixture; and
c) heating the mixture of step b) to a temperature high enough to melt the polymer but not enough to degrade the polymeric material to form the radiolabeled product.

2. The process according to claim 1 wherein the adsorbed radionuclide of step
a) is produced by adding water or an acid/water mixture to the radionuclide and forming a slurry or suspension with the activated charcoal, and then drying the slurry or suspension to form the adsorbed radionuclide.

3. The process according to claim 3 wherein the water/acid mixture uses hydrochloric acid, phosphoric acid or acetic acid.

4. The process according to claim 2 wherein the slurry or suspension is dried in an oven.

5. The process according to claim 1 wherein the insoluble polymer is cellulose acetate, polyvinylacetate, polyethylvinylacetate, polyethylene, polypropylene, polycaprolactone, polyactic acid, polyglycolic acid, and poly(lactic-co-glycolic acid) (PLGA).

6. The process according to claim 5 wherein the insoluble polymer is cellulose acetate.

7. The process according to claim 1 wherein the weight ratio of radiolabeled charcoal of step a) to insoluble polymer is from about 1:3 to about 1:100.

8. The process according to claim 1 wherein the weight ratio of radiolabeled charcoal of step a) to insoluble polymer is from about 1 to about 6.

9. The process according to claim 1 wherein the particle size of the radiolabeled product is about 5 μm and about 20 μm.

10. The process according to claim 1 wherein the radionuclide is indium, samarium, technetium, iodine, and their derivatives or chelate thereof.

11. The process according to claim 10 wherein the radionuclide is indium chloride, samarium oxide, technetium tin colloid, or Pentetate Indium Disodium.

12. The product produced by the process according to claim 1.

13. A process for producing a radiolabeled product which comprises
a) combining a radionuclide with an ion-exchange resin; and
d) reducing the particle size of the product of step a) as desired.

14. The process according to claim 13 wherein the ion exchange resin is selected from Amberjet, Amberlite, Duolite, CM-cellulose or DEAE-cellulose.

15. The process according to claim 13 wherein the radionuclide is indium, samarium, technetium, iodine, and their derivatives or chelate thereof.

16. The process according to claim 15 wherein the radionuclide is indium chloride, samarium oxide, technetium tin colloid, or Pentetate Indium Disodium.

17. The process according to claim 14 wherein the radionuclide is indium, samarium, technetium, iodine, and their derivatives or chelate thereof.

18. The process according to claim 17 wherein the radionuclide is indium chloride, samarium oxide, technetium tin colloid, or Pentetate Indium Disodium.

19. The product produced by the process according to claim 13.

20. A method of producing a radiolabeled gastroretentive formulation (GRF) for use in a human in need thereof, which comprises incorporating a product according to claim 12 into a GRF.

21. The method according to claim 20 wherein the GRF does not prematurely release the radionuclide due to fluctuating pH levels within the stomach environment.

22. A method of producing a radiolabeled gastroretentive formulation (GRF) for use in a human in need thereof, which comprises incorporating into a gastroretentive formulation a radionuclide incorporated into an electrospun fiber, a melt extruded fiber, a melt extruded granule, or an electrosprayed bead.

23. A method of determine the location of a device or formulation in the gastrointestinal tract of a mammal which comprises incorporating a radionuclide into an electrospun fiber, a melt extruded fiber, a melt extruded granule, an electrosprayed bead, or a product according to claim 12.

24. A pharmaceutical composition comprising an effective amount of a radionuclide and activated charcoal.

25. A pharmaceutical composition comprising an effective amount of a radionuclide, activated charcoal, and an insoluble polymer.

26. The composition according to claim 25 wherein the radionuclide is indium, samarium, technetium, iodine, and their derivatives or chelate thereof.

27. The composition according to claim 26 wherein the radionuclide is indium chloride, samarium oxide, technetium tin colloid, or Pentetate Indium Disodium.

28. The composition according to claim 25 wherein the insoluble polymer is cellulose acetate, polyvinylacetate, polyethylvinylacetate, polyethylene, polypropylene, polycaprolactone, polyactic acid, polyglycolic acid, and poly(lactic-co-glycolic acid) (PLGA).

29. The composition according to claim 28 wherein the insoluble polymer is cellulose acetate.

30. The composition according to claim 25 wherein the weight ratio of the radionuclide and charcoal to insoluble polymer is from about 1:3 to about 1:100.

31. The composition according to claim 30 wherein the weight ratio of radionuclide and charcoal to insoluble polymer is from about 1 to about 6.

32. The composition according to claim 25 wherein the particle size of the composition is about 5 μm and about 20 μm.

33. The composition according to claim 25 wherein the radionuclide is indium, samarium, technetium, iodine, and their derivatives or chelate thereof; the insoluble polymer is cellulose acetate, polyvinylacetate, polyethylvinylacetate, polyethylene, polypropylene, polycaprolactone, polylactic acid, polyglycolic acid, and poly(lactic-co-glycolic acid) (PLGA); and the weight ratio of the radionuclide and charcoal to insoluble polymer is from about 1:3 to about 1:100.
